(19) United States
(12) Patent Application Publication
(10) Pub. No.: US 2025/0389628 A1
(43) Pub. Date: Dec. 25, 2025

(54) Pretreatment Method and Mass Spectrometry Method

(71) Applicants: SHIMADZU CORPORATION, Kyoto-shi (JP); NATIONAL INSTITUTE OF ADVANCED INDUSTRIAL SCIENCE AND TECHNOLOGY, Tokyo (JP)

(72) Inventors: Kanae TERAMOTO, Kyoto-shi (JP); Yuji SEKIGUCHI, Tsukuba-shi (JP)

(21) Appl. No.: 19/106,128

(22) PCT Filed: Aug. 24, 2023
(86) PCT No.: PCT/JP2023/030477

§ 371 Date: Feb. 24, 2025

(30) Foreign Application Priority Data
Aug. 26, 2022 (JP) 2022-134981

(51) Int. Cl. G01N 1/40; H01J 49/00; H01J 49/04; H01J 49/16

(52) U.S. Cl. CPC G01N 1/4044; H01J 49/0031; H01J 49/0418; H01J 49/164

(57) ABSTRACT

A method of pretreatment of a sample containing a cell for mass spectrometry, including contacting the cell with a first acidic solution containing an organic acid; and extracting a cytoplasmic component of the cell by heating the cell in contact with the first acidic solution.

### TECHNICAL FIELD

The present invention relates to a pretreatment method and a mass spectrometry method, and more particularly, relates to a pretreatment method and a mass spectrometry method for a sample containing a cell.

### BACKGROUND ART

There is a known method in which a sample containing a cell is pretreated with formic acid before mass spectrometry. NPL 1 discloses that when a prescribed strain is subjected to a formic acid treatment on a target plate or in a microtube before performing mass spectrometry, a result suggesting improvement of identification accuracy is obtained. NPL 2 discloses a pretreatment method for treating, with formic acid, on a sample plate or in a tube, a cell of a prescribed strain having been plate-cultured.

### CITATION LIST

### Non Patent Literature

NPL 1: Comparative study of identification methods for NVS by two different MALDI-TOF MS based devices, VITEK 2 and conventional biochemical test, Michiko Furugaito et al., The Journal of the Japanese Society for Clinical Microbiology, 2016, vol. 26, No. 3, pp. 29-39

NPL 2: Application of MALDI-TOF MS for Rapid Identification of Microorganisms in Food Microbiology, Hiroko Kawasaki, Japanese Journal of Food Microbiology, 2020, vol. 37, No. 4, pp. 165-177

### SUMMARY OF INVENTION

### Technical Problem

As disclosed in NPLs 1 and 2, the method for treating a cell with formic acid is roughly divided into a method in which the treatment is performed on a sample plate, and a method in which the treatment is performed in a container such as a tube.

There is, however, a problem, in a mass spectrum obtained by the method in which the treatment is performed on a sample plate, that the intensity of a peak corresponding to a cytoplasmic component is low. This problem probably reflects that a cytoplasmic component extraction efficiency is low because a cell is not sufficiently destroyed. On the other hand, when the method in which the treatment is performed in a container is employed, respective cells can be more definitely contacted with formic acid, and hence the extraction efficiency of a cytoplasmic component will be probably improved. Actually, it is, however, known that in a microorganism having a strong cell wall, the intensity of a peak corresponding to the cytoplasmic component may be insufficient in some cases even in employing the method in which the treatment is performed in a container.

A ribosomal protein that is a main biomarker for identifying and discriminating a microorganism is contained in the cytoplasm, and hence, means for improving the intensity of a peak of a cytoplasmic component has been demanded.

The present disclosure is devised to solve such a problem, and an object is to improve the intensity of a peak of a cytoplasmic component by a pretreatment of a sample, for mass spectrometry, containing a cell.

### Solution to Problem

A pretreatment method according to a first aspect of the present disclosure is a pretreatment method for a sample, for mass spectrometry, containing a cell, and includes contacting the cell with a first acidic solution containing an organic acid; and extracting a cytoplasmic component of the cell by heating the cell in contact with the first acidic solution.

### Advantageous Effects of Invention

Through a pretreatment of a sample, for mass spectrometry, containing a cell, the intensity of a peak of a cytoplasmic component can be improved.

### BRIEF DESCRIPTION OF DRAWINGS

FIG. 1 is a schematic diagram illustrating the configuration of an analysis device according to an embodiment.

FIG. 2 is a flowchart illustrating a treatment related to an on-plate heating method and a mass spectrometry method.

FIG. 3 is a flowchart illustrating a treatment related to an in-tube heating method and a mass spectrometry method.

FIG. 4 is a diagram illustrating mass spectra obtained at different concentrations of an acid in a first acidic solution.

FIG. 5 is a diagram illustrating mass spectra obtained when a non-heating formic acid treatment is performed.

FIG. 6 is a diagram illustrating mass spectra obtained when a heating formic acid treatment is performed.

FIG. 7 is a diagram illustrating intensity change of a ribosome peak caused by the heating formic acid treatment.

FIG. 8 is a diagram illustrating mass spectrum change caused by the heating formic acid treatment in a microorganism different from that of FIG. 7.

FIG. 9 is a diagram illustrating the number of peaks obtained when a formic acid treatment is performed on Escherichia coli under a plurality of temperature conditions.

FIG. 10 is a diagram illustrating the number of peaks obtained when a formic acid treatment is performed on Janibacter limosus under a plurality of temperature conditions.

FIG. 11 is a diagram illustrating mass spectrum change caused by the heating formic acid treatment performed on Aspergillus kawachii.

### DESCRIPTION OF EMBODIMENTS

Now, one embodiment of the present disclosure (hereinafter referred to as the “present embodiment”) will be described. It is noted that the present embodiment is not limited to the following. Herein, the expression in the form of “A to Z” means upper and lower limits of a range (namely, A or more and Z or less), and when A does not have a unit but only Z has a unit, the unit of A is the same as the unit of Z.

Moreover, herein, “%” used for a solution means “vol %” unless otherwise stated.

The present embodiment will now be described in detail with reference to the accompanying drawings. It is noted that in the following description, the same or corresponding components are referred to with the same reference signs in the drawings to basically avoid redundant description.

### 1. Configuration of Analysis Device

First, an example of an analysis device 1 for practicing a mass spectrometry method of the present embodiment will be described. The mass spectrometry method of the present embodiment includes a biological sample pretreatment method of the present embodiment. It is noted that a “pretreatment” herein refers to preparation of a biological sample performed before mass spectrometry unless otherwise stated.

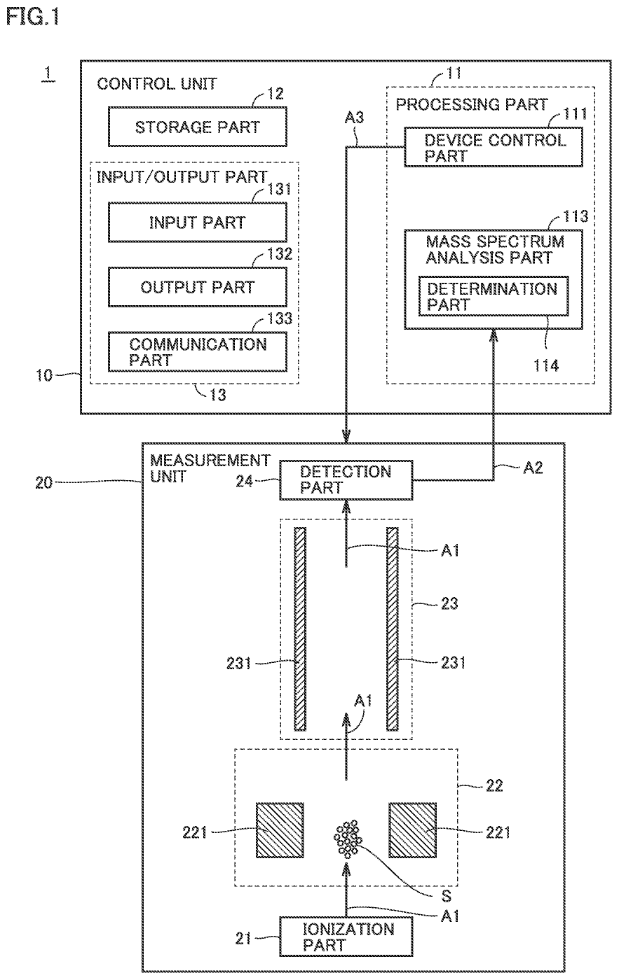

FIG. 1 is a schematic diagram illustrating the configuration of analysis device 1. Analysis device 1 is a mass spectrometer for performing mass spectrometry of a substance contained in a sample, and is, for example, MALDI-TOF MS (Matrix-Assisted Laser Desorption/Ionization Time-of-Flight Mass Spectrometry). Analysis device 1 determines the type of an organism by using a mass spectrum obtained by mass spectrometry.

The sample contains a cell of an organism. The cell contains a target substance, that is, a molecule to be analyzed. Moreover, in the present embodiment, the analysis with analysis device 1 includes detecting a peak of a mass spectrum, and measuring a mass-to-charge ratio (m/z) of a specific or nonspecific substance contained in the sample. In one example, the substance is a protein. The analysis with analysis device 1 includes determining, based on a m/z corresponding to a peak of the mass spectrum, the type of a microorganism from which the sample is derived. The m/z corresponding to a peak of a mass spectrum is generally referred to also as the “position” or “m/z position” of the peak.

Herein, the type of a microorganism includes at least one of taxonomic classes of the genotype, the strain, the subspecies, the species, the genus, and the family of a microorganism, unless otherwise stated. Moreover, the determination of the type of a microorganism includes classification, identification, and discrimination of the type of the microorganism. Hereinafter, the determination of the type of a microorganism is referred to simply as determination of the microorganism.

The analysis with analysis device 1 may include determining whether or not a specific substance is contained in the sample.

Referring to FIG. 1, analysis device 1 includes a control unit 10 and a measurement unit 20.

Measurement unit 20 ionizes, with a high voltage, a substance (e.g., protein) contained in a sample, and detects the resultant ion S after separation in accordance with time of flight correlated with a m/z. Measurement unit 20 includes an ionization part 21, an ion acceleration part 22, a mass separation part 23, and a detection part 24. In FIG. 1, the movement of the ion S in measurement unit 20 is schematically illustrated with an arrow A1.

In one example, ionization part 21 ionizes the substance contained in the sample by a matrix-assisted laser desorption/ionization (MALDI) method. The MALDI method is a useful method in determination of a microorganism by mass spectrometry of the microorganism as described below. As the ionization method, not only the MALDI method but also any soft ionization method such as an electrospray ionization (ESI) method can be employed. In the ionization performed by the ESI method, a configuration in which analysis device 1 further includes a liquid chromatograph for ionizing, with ionization part 21, a substance that is contained in the sample, and has been separated with the liquid chromatograph is preferred because high separability can be thus obtained.

Ionization part 21 includes a sample plate holder (not shown) for holding a sample plate, and an ion source including a laser device (not shown) for irradiating the sample plate with a laser beam. An analyzer mixes a matrix solution with the sample having been subjected to the pretreatment method of the present embodiment described below, and places the resultant mixture on the sample plate. In the matrix solution, a matrix substance that easily absorbs a laser beam, and is easily ionized with the laser beam is contained. The matrix substance can be, but is not limited to, for example, α-cyano-4-hydroxycinnamic acid (4-CHCA), α-cyano-3-hydroxycinnamic acid (3-CHCA), sinapinic acid, ferulic acid, 3-hydroxy-4-nitrobenzoic acid (3H4NBA), 2,5-dihydroxybenzoic acid, or 1,5-diaminonaphthalene.

The analyzer obtains a dried product by drying, on the sample plate, a sample mixed matrix solution obtained as described above by mixing the sample with the matrix solution. Thereafter, the sample plate is set on the sample plate holder disposed in a vacuum container of ionization part 21.

It is noted that the dried product obtained by mixing the sample with the matrix solution and drying the resultant is generally referred to also as the “crystal” in some cases, and more specifically, is variously referred to as a “crystal”, “mixed crystal”, “sample crystal”, “matrix crystal”, “sample/matrix crystal”, and the like. Herein, the dried product will be hereinafter referred to as the “matrix dried product”.

In the following description, the sample mixed matrix solution having been placed on the sample plate to be formed into the matrix dried product is described as including the matrix solution with which the sample has been mixed, and will be referred to as the “matrix solution”, unless otherwise stated.

Ionization part 21 depressurizes the vacuum container in which the sample plate has been set, and then irradiates the matrix dried product on the sample plate with a laser beam for ionization of the target substance contained in the matrix dried product. The type of the laser device for emitting the laser beam is not especially limited as long as it can oscillate light absorbed by the used matrix solution, and for example, when the matrix solution contains CHCA, N2 laser (wavelength of 337 nm) or the like can be suitably used. An ion S of the target substance having been ionized by ionization part 21 is extracted from an electric field formed by an extraction electrode or the like not shown, and is introduced into ion acceleration part 22.

Ion acceleration part 22 includes an accelerating electrode 221, and accelerates the ion S having been introduced thereinto. The flow of the accelerated ion S is appropriately converged by an ion lens, which is not shown, to be introduced into mass separation part 23.

Mass separation part 23 includes a flight tube 231, and separates ions S in accordance with a difference in time of flight spent by the respective ions S flying inside flight tube 231. Although FIG. 1 illustrates linear flight tube 231, a reflectron flight tube, a multi-turn flight tube or the like may be used. The mass spectrometry method is not especially limited as long as ions S contained in the sample can be separated and detected.

Detection part 24 includes an ion detector such as a multi-channel plate, detects the ion S separated by mass separation part 23, and outputs a detected signal with an intensity according to the number of ions having entered detection part 24. The detected signal output from detection part 24 is input to a processing part 11 of control unit 10. In FIG. 1, a flow of the detected signal of the ions S from detection part 24 of measurement unit 20 is schematically illustrated with an arrow A2.

Control unit 10 includes processing part 11, a storage part 12, and an input/output part 13. Control unit 10 is configured, for example, by one or a plurality of computers.

Processing part 11 is configured by including a processor such as a CPU, and functions as a main part in an operation for controlling analysis device 1. Processing part 11 performs various processing by executing a program stored in storage part 12 and the like.

Processing part 11 includes a device control part 111 and a mass spectrum analysis part 113. Mass spectrum analysis part 113 includes a determination part 114.

Device control part 111 controls the operation of measurement unit 20 based on data related to analysis conditions input from an input part 131 described below. In FIG. 1, the control of measurement unit 20 by device control part 111 is schematically illustrated with an arrow A3.

Mass spectrum creation part 113 converts the time of flight into a m/z based on measurement data including the amount of ions detected by detection part 24, and the time of flight of the ions, and creates a mass spectrum indicating a detection amount corresponding to each m/z.

The number of detected signals of the target substance detected by detection part 24, and the intensities of the detected signals correlate with the number of peaks corresponding to the target substance in the mass spectrum, and the intensities of the peaks. In other words, there is a relationship in which as the amount of the target substance contained in the matrix dried product is larger, the intensity of the peak is higher. Therefore, there is a relationship in which as the extraction efficiency of the target substance in the sample pretreatment is higher, the intensity of the peak is higher.

Mass spectrum analysis part 113 further obtains a m/z corresponding to the peak of the mass spectrum. Mass spectrum analysis part 113 may determine, based on protein database or the like, a substance corresponding to a m/z indicated by the peak of the mass spectrum. In other words, mass spectrum analysis part 113 can calculate a m/z of a specific or nonspecific substance contained in the sample. Mass spectrum analysis part 113 may further determine, based on the m/z, whether or not the specific substance is contained in the sample (component identification in the sample).

Mass spectrum analysis part 113 includes determination part 114. In one example, determination part 114 creates database including mass spectra, and stores it in storage part 12. The database includes one or more, and preferably a large number of mass spectra of microorganisms of known types. Determination part 114 determines a microorganism using the database of mass spectra.

In one example, determination part 114 determines a microorganism by a fingerprint method. Specifically, determination part 114 determines a microorganism by comparing the pattern of a mass spectrum of an unknown microorganism with the pattern of a mass spectrum of a known microorganism stored in the database. The determination of a microorganism is performed by referring to a peak of a biomarker that is a substance showing an expression pattern characteristic to each microorganism. In a microorganism, a ribosomal protein is mainly used as a biomarker.

Storage part 12 includes a nonvolatile storage medium. Storage part 12 stores the mass spectrum created by mass spectrum creation part 113, the measurement data output from measurement unit 20, the program used for executing processing by processing part 11, and the like. Storage part 12 corresponds to an example of a “memory” of the present disclosure. Storage part 12 may include a storage medium removable from analysis device 1. The storage medium may be any medium capable of storing various data, such as a CD (compact disc), a DVD (digital versatile disc), and a USB (universal serial bus) memory. In one example, storage part 12 stores database including the mass spectrum thus obtained.

Input/output part 13 is an interface for inputting/outputting information between analysis device 1 and the outside. Input/output part 13 includes an input part 131, an output part 132, and a communication part 133.

Input part 131 is configured by including an input device such as a mouse, a keyboard, various buttons and/or a touch panel. Input part 131 receives, from an analyzer, information necessary for control of the operation of measurement unit 20, and information necessary for processing performed by processing part 11, and the like.

Output part 132 is configured by including a display device such as a liquid crystal monitor, a printer, and the like. Output part 132 displays, in a display device, information on the measurement by measurement unit 20, and results of the processing by processing part 11, or prints these on print media.

Communication part 133 is configured by including a communication device capable of communication through wireless or wired connection such as Internet. Communication part 133 receives data necessary for the processing by processing part 11, transmits data having been processed by processing part 11, such as determination results, and appropriately receives/transmits necessary data.

A part or the whole of the function of control unit 10 described above may be disposed in a computer, a server, or the like physically separated from measurement unit 20.

Analysis device 1 used in the present embodiment is preferably, but not limited to, a device combined with a MALDI (matrix-assisted laser desorption/ionization) ion source. Examples of the device combined with a MALDI ion source include a MALDI-IT (matrix-assisted laser desorption/ionization-ion trap) mass spectrometer, a MALDI-IT-TOF (matrix-assisted laser desorption/ionization-ion trap-time of flight) mass spectrometer, and a MALDI-FTICR (matrix-assisted laser desorption/ionization-Fourier transform ion cyclotron resonance) mass spectrometer. Moreover, analysis conditions employed in analysis device 1 are set within a range usually set by those skilled in the art.

### 2. Conventional Sample Pretreatment Method

Conventionally, there is a method for determining a microorganism by mass spectrometry. In mass spectrometry, a mass spectrum corresponding to an analysis result can be obtained easily and in a short period of time by using a very small amount of a microorganism sample. Moreover, analysis of multiple samples can be performed rapidly and easily by employing automatic analysis.

In such mass spectrometry, in particular, analysis of a microorganism by MALDI-MS, that is, one of soft ionization methods for ionizing a biopolymer such as a protein substantially without degrading it, is widely utilized. Specifically, prescribed types of microorganisms are determined by MALDI-MS in the fields of clinical microorganism analysis, food safety test and the like. In this manner, MALDI-MS is currently a very excellent technique in the determination of prescribed types of microorganisms, but may be difficult to employ for determination of a specific type of microorganisms in some cases.

For example, the simplest, and frequently employed pretreatment method can be a method of simply mixing a cell and a matrix solution. When this simplest pretreatment method is used for a microorganism having a strong cell wall (e.g., a gram-positive bacterium, or a fungus), however, a peak of a ribosomal protein may not be detected with sufficient intensity in a mass spectrum in some cases. This result probably reflects that the cell wall of the microorganism is not sufficiently destroyed by simply mixing with the matrix solution, and hence a component present inside the cell wall and/or the cell membrane, including a ribosomal protein, does not flow out. Hereinafter, the “component present inside the cell wall and/or the cell membrane” will be referred to as the “intracellular component”. The intracellular component includes a cytoplasmic component and a nuclear component, and a ribosomal protein is included in the cytoplasmic component.

For a ribosomal protein, a small difference in the amino acid sequence, and a resultant difference in the mass can be a definite index for evaluating a difference in the species of an organism, and hence it is used as a biomarker for identifying a species. Besides, a ribosomal protein is a structure present in a large amount within a cell, and hence is easily detected in mass spectrometry.

Besides, most of ribosomal proteins are basic proteins having high proton affinity, and hence easily generate [M+H]⁺ ions in the MALDI process. Furthermore, a ribosomal protein has a molecular weight of about 5000 to 20000, and the mass of a ribosomal protein can be obtained within the margin of error of several Da by MALDI-MS. Accordingly, MALDI-MS employed as the mass spectrometry has a merit that a peak of a ribosomal protein can be easily detected in a sample.

As described so far, a ribosomal protein is a very excellent biomarker, and hence, is generally mainly used as a biomarker in determination of a microorganism by mass spectrometry.

Besides, although a ribosomal protein is mainly used in the determination of a microorganism as described above, it is known that a DNA binding protein, an RNA binding protein, and a molecular chaperone are also usable. These proteins are also contained in an intracellular component in the same manner as a ribosomal protein.

Besides, when mass spectrometry is performed by employing the simplest pretreatment method described above, the number and/or intensities of peaks of biomarkers, such as a ribosomal protein, are not sufficient in some types of microorganisms, and hence the determination may be difficult in some cases.

In order to extract a cytoplasmic component from such a cell having a strong cell wall to obtain peaks in a sufficient number and/or with sufficient intensities, a pretreatment method with formic acid is known.

NPLs 1 and 2 respectively disclose a method for treating a cell with formic acid on a sample plate, and a method for treating a cell with formic acid in a tube. Hereinafter, for comparison with a formic acid treatment accompanied by heating according to an embodiment described below, the conventional formic acid treatment performed on a sample plate is referred to also as the “on-plate non-heating treatment”, and the conventional formic acid treatment performed in a tube is referred to also as the “in-tube non-heating treatment”. Besides, a pretreatment method by the “on-plate non-heating treatment” is referred to also as an “on-plate non-heating method”, and a pretreatment method by the “in-tube non-heating treatment” is referred to also as an “in-tube non-heating method”.

In the on-plate non-heating method, a matrix dried product is obtained by, for example, dropping formic acid onto a microorganism cell applied on a sample plate, performing the on-plate non-heating treatment, drying the resultant, dropping a matrix solution onto the resultant, and drying the resultant again.

In the in-tube non-heating method, the in-tube non-heating treatment is performed by mixing formic acid with a cell within a tube, and then, acetonitrile is further added to the tube to be mixed in many cases. Thereafter, the tube is centrifuged, and a supernatant is dropped onto a sample plate to be dried. Then, a matrix solution is dropped thereon, and the resultant is dried again, and thus, a matrix dried product is obtained.

In the in-tube non-heating method, the cell may be dispersed in ethanol, and the resultant may be centrifuged to remove a supernatant before adding formic acid thereto.

The on-plate non-heating method has, however, a problem that the peak intensity of a cytoplasmic component is low, and is not sufficient for determination of a microorganism in some cases as compared with the in-tube non-heating method.

In the in-tube non-heating method, a sample and formic acid are mixed in a tube, and hence the extraction efficiency is probably improved as compared with that in the on-plate non-heating method. In a gram-positive bacterium (e.g., Mycobacterium tuberculosis) or a fungus (e.g., mold or yeast) having a strong cell wall, however, a sufficient peak intensity cannot be obtained in some cases. In particular, there is a problem that the peak intensity is low in a gram-positive bacterium having, in the cell wall, a long chain fatty acid designated as mycolic acid (for example, an actinomycete belonging to the suborder Corynebacterineae, such as Mycobacterium and Nocardia). In other words, it is presumed that the cytoplasm extraction efficiency is not sufficient even in employing the in-tube non-heating treatment. This is probably because the layer of fatty acid is thick in a gram-positive bacterium having mycolic acid in the cell wall.

The group of bacteria containing mycolic acid encompasses Mycobacterium tuberculosis and non-tuberculous mycobacteria that are significant pathogenic bacteria. As the same mycolic acid-producing bacteria, bacteria belonging to the genus Nocardia are significant pathogenic bacteria as causative bacteria of infectious diseases of the skin and central nervous system (nocardiosis). Therefore, it is significant in the field of medical and clinical research to appropriately determine a gram-positive bacterium containing mycolic acid in the cell wall.

On the other hand, as a pretreatment method for disrupting (lysing) a cell by a method except for the chemical reaction with formic acid, a bead grinding treatment is also known. In the bead grinding treatment, a tube is charged with a cell, a solvent, and a bead (e.g., 0.5 mm zirconia bead), followed by shaking, and thus, the cell is disrupted by physical friction. In this manner, the extraction efficiency of a cytoplasmic component is probably improved.

In the bead grinding treatment, however, the bead may clog in or be sucked into a pipette tip in some cases, and hence, this method may be troublesome in handling. Besides, if the bead is mixed with a matrix dried product, it is apprehended that the bead may be peeled off from the sample plate within a device to adhere to or enter the device. For example, there is a possibility that the bead may fall out the matrix dried product by impact with laser irradiation. In this case, there is a possibility that a defect or failure of the device may be caused.

As described so far, in the pretreatment method with formic acid, a sufficient peak intensity of a cytoplasmic component cannot be obtained in some cases depending on the type of microorganism. Besides, the bead grinding treatment has a problem of causing a failure of the device, and the like. Therefore, there is a demand, in pretreatment of a sample, for mass spectrometry, containing a cell, for a method for improving a peak intensity of a cytoplasmic component without using a bead.

### 3. Sample Pretreatment Method of Embodiment

In consideration of these circumstances, the present inventors have found, as a result of trial and error on a sample pretreatment, a method for improving a peak intensity of a cytoplasmic component without using a bead.

In a pretreatment method of the present embodiment, heating is performed after mixing a microorganism with an acidic solution. Through this heating, the peak intensity of a cytoplasmic component is improved.

More specifically, the pretreatment method of the present embodiment is a method of pretreatment of a sample containing a cell for mass spectrometry, including a “contacting process” for contacting the cell with a first acidic solution containing an acid, and a “heating process” for heating the cell in contact with the first acidic solution.

### (3-1. Type of Microorganism)

A cell of a microorganism contained in the sample is not limited to a cell of a microorganism having a gram-negative cell wall structure but may be a cell of a microorganism having a gram-positive cell wall structure. More specifically, the cell of the microorganism contained in the sample may be a gram-negative bacterium (e.g., E. coli), a gram-positive bacterium (e.g., Mycobacterium tuberculosis), or a fungus (e.g., mold or yeast).

In more detail, the sample may contain a prokaryote-derived cell, or may contain a eukaryote-derived cell. The sample is typically derived from a microorganism, and may contain an unknown microorganism. The prokaryote includes bacteria and archaea. Examples of the bacteria include bacteria belonging to the genus Escherichia (e.g., E. coli), the genus Bacillus (e.g., Bacillus subtilis), the genus Lactobacillus (e.g., lactic acid bacteria), the genus Synechocystis (e.g., cyanobacteria), and the genus Mycobacterium (e.g., actinomycetes). Examples of the archaea include the genus Methanophilus, the genus Methanococcus, the genus Thermococcus, and the genus Phyllococcus. The eukaryote includes animals, plants, fungi, and protists. The fungi include filamentous fungi, yeasts, mushrooms, molds, and the like, and encompass the phylum Chytridiomycota, the phylum Zygomycota, the phylum Ascomycota, the phylum Basidiomycota, the phylum Glomeromycota, the phylum Microsporidia, and the like. The phylum Ascomycota includes the genus Aspergillus (e.g., Aspergillus oryzae), the genus Penicillium (e.g., Penicillium chrysogenum), the genus Saccharomyces (e.g., budding yeast), and the like. In one example, the sample is a cell of a bacterium (bacterial cell), in a broad sense, including the above-described bacteria, archaea, and fungi.

Through the following two processes, cells of these microorganisms are destroyed, and a cytoplasmic component is eluted therefrom.

### (3-2. Contacting Process)

The sample to be contacted with the first acidic solution may be in a liquid form or a solid. More specifically, the sample may be, for example, a liquid containing a culture fluid of a bacterium, or may be a scrape, obtained with a toothpick or the like, of a colony of a bacterium having been cultured on a solid medium. The culture fluid of the bacterium contains the bacterium, and a medium used for culturing the bacterium.

The acid is, for example, an organic acid, such as formic acid, trifluoroacetic acid, or acetic acid, generally used in a biological experiment. These organic acids are easily available for and familiar in handling to users of the research and clinical fields performing mass spectrometry of microorganisms. Besides, these organic acids do not harmfully affect a measurement result even when used in a pretreatment of mass spectrometry by those skilled in the art. Another example of the acid includes an acid having an acid dissociation constant (pKa) of 0.2 or more and 5 or less, and more preferably an acid having an acid dissociation constant of 0.2 or more and 4 or less. The acid having an acid dissociation constant of 0.2 or more and 5 or less is, for example, at least one acid out of trifluoroacetic acid, oxalic acid, glycine, salicylic acid, formic acid, lactic acid, benzoic acid, acetic acid, and butyric acid, some of which are the same as those described above as the examples of the acid. It goes without saying that the examples of the acid are not limited these. In one example, the organic acid is formic acid. Besides, as described below, an inorganic acid is similarly used. Furthermore, the acid contained in the first acidic solution may be one acid (pure substance), or may be a mixture of a plurality of acids.

Herein, the “acid” refers to a pure substance (for example, 100% formic acid). Besides, a solution obtained by diluting the acid to a prescribed concentration with a solvent is referred to as the “first acidic solution”. The solvent typically includes water. Water is preferably pure water, ultrapure water, or ion exchange water. The solvent may contain an organic solvent in addition to water or instead of water as long as the effects of the pretreatment method of the present embodiment are exhibited. The organic solvent is, for example, an organic solvent having a polarity. Examples of the organic solvent include acetonitrile, methanol, and ethanol.

As specific means for the “contacting process”, means for mixing the sample and the first acidic solution is typically used. Herein, a mixture solution of the sample and the first acidic solution is referred to as the “second acidic solution”. By mixing the sample and the first acidic solution, the cell can be dispersed in the first acidic solution. Thus, the cell can be definitely contacted with the first acidic solution, and hence, the efficiency of the treatment of the cell with the first acidic solution is improved.

A mixing ratio between the first acidic solution and the sample is not limited as long as the effects of the pretreatment method of the present embodiment are exhibited, and for example, the sample is mixed in an amount of 1/2 or less and 1/1000 or more, in terms of a volume ratio, of the first acidic solution. In general, however, the amount of components contained in the sample in addition to the cell (for example, a liquid medium used for culturing the cell) is reduced as much as possible in mixing with the first acidic solution. Specifically, for example, the sample is centrifuged before being mixed with the first acidic solution, a supernatant is removed, a remaining precipitate is collected, and the collected precipitate is mixed with the first acidic solution. It is more preferable that the precipitate is centrifugally washed with ultrapure water or the like. For these reasons, a content ratio (concentration) of the acid in the second acidic solution, and the pH thereof are substantially not changed from those of the first acidic solution. Hereinafter, the first acidic solution and the second acidic solution are generically designated as the “acidic solution” in some cases.

The concentration of the acid in the acidic solution is, for example, 50 to 90 vol %, preferably 60 to 80 vol %, and more preferably 65 to 75 vol %.

In one example, the first acidic solution is 50 to 90% formic acid, preferably 60 to 80% formic acid, and more preferably 65 to 75% formic acid. In this example, the second acidic solution is formic acid containing the cell and having any of these concentrations.

In one example, the first acidic solution is 50 to 90% trifluoroacetic acid, preferably 60 to 80% trifluoroacetic acid, and more preferably 65 to 75% trifluoroacetic acid. In this example, the second acidic solution is trifluoroacetic acid containing the cell and having any of these concentrations.

In one example, the first acidic solution is 50 to 90% acetic acid, preferably 60 to 80% acetic acid, and more preferably 65 to 75% acetic acid. In this example, the second acidic solution is acetic acid containing the cell and having any of these concentrations.

It can be confirmed whether or not the acidic solution has been correctly mixed by measuring a hydrogen ion exponent (pH) of the acidic solution with, for example, a PH meter.

Another example of the specific means for the “contacting process” is means for contacting (e.g., placing) the cell with or on a material (e.g., cloth) soaked with the first acidic solution. In this manner, the specific means for the “contacting process” is not limited as long as the effects of the pretreatment method of the present embodiment are exhibited.

Next, two pretreatment methods included in the pretreatment method of the present embodiment will be successively described. These two pretreatment methods are different in the heating method. The heating method will be described below.

### (3-3. Heating Process)

The heating process is performed for improving the peak intensity of a cytoplasmic component. More specifically, it is presumed that cell destruction efficiency is improved by heating the second acidic solution in the heating process, and thus, the extraction efficiency of a cytoplasmic component is improved.

A heating time is, for example, 2 minutes or more and less than 20 minutes, preferably 3 minutes or more and 15 minutes or less, and more preferably 5 minutes or more and 10 minutes or less.

A heating temperature is, for example, 30 to 75° C., preferably 40 to 60° C., more preferably 45 to 55° C., and further preferably about 50° C. In one aspect of the present embodiment, the heating temperature may be 35 to 55° C., and may be about 40 to about 50° C.

### (3-4. On-plate Heating Method)

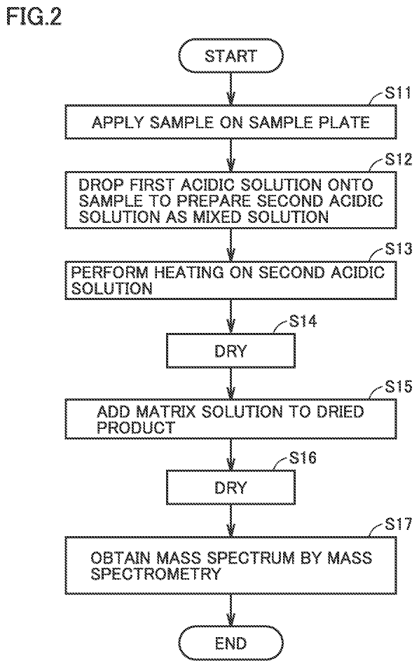

The pretreatment method of the present embodiment includes a pretreatment method including a treatment for heating the second acidic solution placed on a sample plate. Hereinafter, the treatment for heating the second acidic solution placed on a sample plate will be referred to as the “on-plate heating treatment”, and a pretreatment method including the on-plate heating treatment will be referred to as the “on-plate heating method”. Next, the on-plate heating method will be described with reference to FIG. 2.

FIG. 2 is a flowchart illustrating a treatment related to the on-plate heating method and a mass spectrometry method. The on-plate heating method illustrated in FIG. 2 is one embodiment of the pretreatment method of the present embodiment. Steps illustrated in FIG. 2 are performed manually by an analyzer with experimental tools and experimental devices used in a general pretreatment and mass spectrometry of a microorganism. In the drawing, “S” is used as an abbreviation of “STEP”.

In S11, an analyzer applies a sample to a sample plate. For example, the analyzer scrapes a colony of a bacterium on a solid medium with a toothpick, and applies it to the sample plate.

In S12, the analyzer drops a first acidic solution onto the applied sample, and thus prepares a second acidic solution that is a mixture solution of the sample and the first acidic solution. The mixing is performed, for example, by performing pipetting with a micropipette a plurality of times. S12 corresponds to one embodiment of the “contacting process”.

In S13, the analyzer performs heating on the second acidic solution. The heating is performed by, for example, holding, for a prescribed period of time, the sample plate in an incubator set to a prescribed temperature. The treatment of S3 corresponds to the “on-plate heating treatment”. Besides, it corresponds to one embodiment of the “heating process”.

In S14, the analyzer dries the second acidic solution on the sample plate.

In S15, the analyzer adds a matrix solution to a dried product obtained by drying the second acidic solution (hereinafter referred to also as the “acid dried product”). For example, the analyzer drops the matrix solution within several minutes after the second acidic solution has dried, and mixes the acid dried product with the matrix solution by performing pipetting with a micropipette a plurality of times.

In S16, the analyzer obtains a matrix dried product by drying the matrix solution containing the sample on the sample plate.

In S17, the analyzer obtains a mass spectrum by placing the sample plate in a mass spectrometer, and performing mass spectrometry. The mass spectrometry is preferably MALDI mass spectrometry.

### (3-5. In-tube Heating Method)

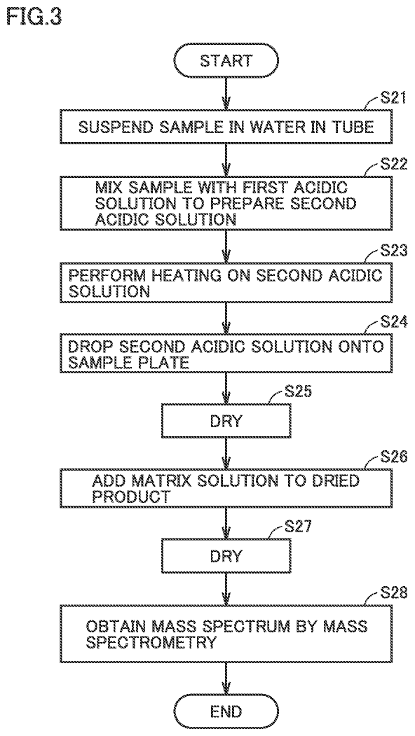

The pretreatment method of the present embodiment includes a pretreatment method including a treatment for heating a second acidic solution held in a container such as a tube before placing it on a sample plate. Hereinafter, the treatment for heating a second acidic solution held in a container will be referred to as the “in-tube heating treatment”, and the pretreatment method including the in-tube heating treatment will be referred to as the “in-tube heating method”. Next, the in-tube heating method will be described with reference to FIG. 3.

FIG. 3 is a flowchart illustrating a treatment related to the in-tube heating method and a mass spectrometry method. The in-tube heating method illustrated in FIG. 3 is one embodiment of the pretreatment method of the present embodiment. Steps illustrated in FIG. 3 are performed manually by an analyzer with experimental tools and experimental devices used in a general pretreatment and mass spectrometry of a microorganism.

In S21, an analyzer suspends, in a tube, a sample containing a cell in water. For example, the analyzer scrapes a colony cultured in a solid medium with a quantitation loop or the like, adds it to a 1.5 mL tube holding 200 μL of water, and mixes the resultant with a vortex mixer for 30 seconds. More specifically, the analyzer extracts a colony having a diameter of about 3 mm with a platinum loop five times or more, and suspends about 10 mg of the bacterial cell in water.

After suspending the sample in water, the sample may be washed by adding ethanol thereto if necessary. The sample washing with ethanol has an effect of dispersing, in a solution, cells that have been aggregated due to secretion and the like of the microorganism. In the pretreatment method of the present embodiment, however, a peak with a sufficient intensity can be obtained without the sample washing with ethanol, and hence, the sample washing is not essential.

Now, an exemplified case in which the sample washing with ethanol is performed will be described. In the washing with ethanol, the concentration of ethanol is preferably 80% or more based on the mixture of the sample and ethanol. The analyzer first adds 800 μL of ethanol to a tube holding a sample (for example, a tube holding a suspension containing about 10 mg of a bacterial cell suspended in 200 μL of water), and mixes the resultant with a vortex mixer for 30 seconds. Next, the analyzer centrifuges the resultant mixture at 10000 g for 2 minutes, and removes a supernatant as much as possible. The analyzer may perform the centrifugation again to completely remove a supernatant if necessary. After removing the supernatant, ethanol is preferably vaporized by naturally drying the resultant for several minutes.

In S22, the analyzer mixes, within the tube, the sample with a first acidic solution to prepare a second acidic solution that is a mixture solution. For example, the analyzer adds 50 μL of the first acidic solution to a precipitate containing the sample in the tube, and mixes the resultant with a vortex mixer for 10 seconds. S22 corresponds to one embodiment of the “contacting process”.

In S23, the analyzer performs heating on the second acidic solution. The heating is performed by, for example, holding, in a PCR (polymerase chain reaction) device set to a prescribed temperature, the tube holding the second acidic solution for a prescribed period of time. The treatment of S24 corresponds to the “in-tube heating treatment”. Besides, it corresponds to one embodiment of the “heating process”.

After S23, the analyzer may perform a treatment for adding an organic solvent such as acetonitrile to the second acidic solution. The organic solvent can be used for the purpose of dissolving a matrix substance contained in a matrix solution, adjusting the surface tension of the matrix solution, destroying the cell membrane, and the like. In the pretreatment method of the present embodiment, however, a peak with a sufficient intensity can be obtained without performing the treatment for adding the organic acid, and hence the treatment for adding the organic solvent is not essential.

It is noted that the treatment for adding the organic solvent is performed, for example, as follows. The analyzer adds, to the tube, acetonitrile in the same amount as formic acid, and mixes the resultant with a vortex mixer for 10 seconds.

In S24, the analyzer drops the second acidic solution onto a sample plate. For example, the analyzer drops, onto the sample plate, 0.5 to 1 μL of a supernatant obtained by centrifuging, after the heating, the tube at 10000 to 15000 g for 2 minutes.

Treatments in S25 to S28 respectively correspond to the treatments of S14 to S17 of FIG. 3, and hence will not be described redundantly.

In the mass spectrum of the sample having been pretreated by the pretreatment method of the present embodiment described so far, the intensity of a peak of an intracellular component is improved. As a result, cell destruction efficiency is probably improved by the pretreatment method of the present embodiment, which probably improves extraction efficiency of the intracellular component. Besides, improvement of determination efficiency of a microorganism by using a peak of a biomarker contained in the intracellular component can be expected.

### 4. Experimental Examples

Next, the pretreatment method of the present embodiment and the effects thereof will be described in more detail with reference to experimental examples, and it is noted that the pretreatment method of the present embodiment is not limited to the experimental examples.

### (4-1. Experiment 1)

Experiment 1 is an experiment showing the influence of the concentration of an acid in a first acidic solution.

A sample pretreatment of Experiment 1 was performed as follows.
(1) A sample containing a mycolic acid-producing bacterium, Rhodococcus erythropolis NBRC 15567T, was dispersed in 500 μL of ultrapure water in a microtube.
(2) The bacterial cell thus dispersed in ultrapure water was dispensed by 100 μL.
(3) To each microtube into which the bacterial cell had been dispensed, 400 μL of ethanol was added to be mixed, and the resultant was centrifuged to obtain a precipitate of the bacterial cell.
(4) To each precipitate obtained by removing a supernatant, 50 μL of 50% formic acid, 50 μL of 70% formic acid, or 50 μL of 90% formic acid was added, followed by mixing with a vortex mixer. Thereafter, the sample held in each tube was subjected to the following treatments.
(5) Heating was performed at 50° C. for 5 minutes.
(6) 50 μL of acetonitrile was further added thereto, and the resultant was mixed with a vortex mixer, and centrifuged to obtain a supernatant.
(7) 1 μL of the supernatant was dropped onto a sample plate and dried thereon, and 1 μL of a matrix solution (CHCA solution) was then dropped and dried thereon, and thus, a matrix dried product was prepared to be subjected to measurement by MALDI-MS.

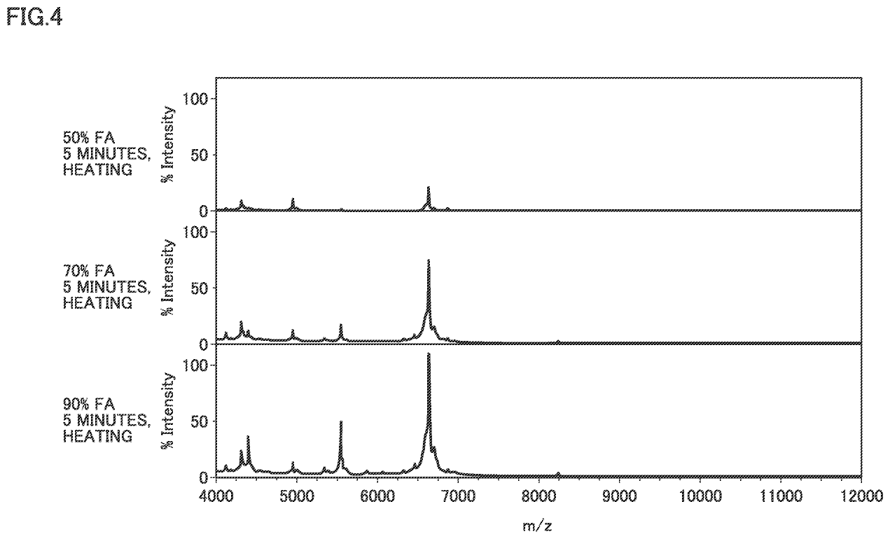

FIG. 4 is a diagram illustrating mass spectra, resulting from Experiment 1, obtained at different concentrations of the acid in the first acidic solution. In FIG. 4, the abscissa indicates the m/z. The ordinate indicates the % intensity, that is, a relative intensity obtained by assuming that one peak having the highest intensity in the three mass spectra of FIG. 4 is 100%.

It is noted that “X % FA” illustrated in FIG. 4 means a first acidic solution containing X % of formic acid. Besides, in the description referring to FIG. 4, a mass spectrum obtained as a result of heating a first acidic solution containing X % of formic acid is referred to also as the “X % formic acid mass spectrum”.

Referring to FIG. 4, the peak intensity was increased by several times to several tens times in the 70% formic acid mass spectrum and the 90% formic acid mass spectrum as compared with that in the 50% formic acid mass spectrum. For example, a peak in the vicinity of a m/z of 6600 was increased in the peak intensity by 3 to 5 times. Besides, as for a peak in the vicinity of a m/z of 5500, substantially no peak was detected in the 50% formic acid mass spectrum, but definite peaks were detected in the 70% formic acid mass spectrum and the 90% formic acid mass spectrum, and the intensity was higher by about 8 to 20 or more times.

In other words, according to the results of Experiment 1, the peak intensity was improved by heating with 70 to 90% formic acid used as compared with that using 50% formic acid. Therefore, it is probably more preferable that 70 to 90% formic acid is used in heating.

In considering that formic acid has a distinctive smell, it is presumed that the 70% formic acid having a weaker smell than the 90% formic acid is further preferably used.

### (4-2. Experiment 2)

Experiment 2 is an experiment showing the influence of a heating time.

In a sample pretreatment of Experiment 2, (4) and (5) of the sample pretreatment of Experiment 1 were modified.

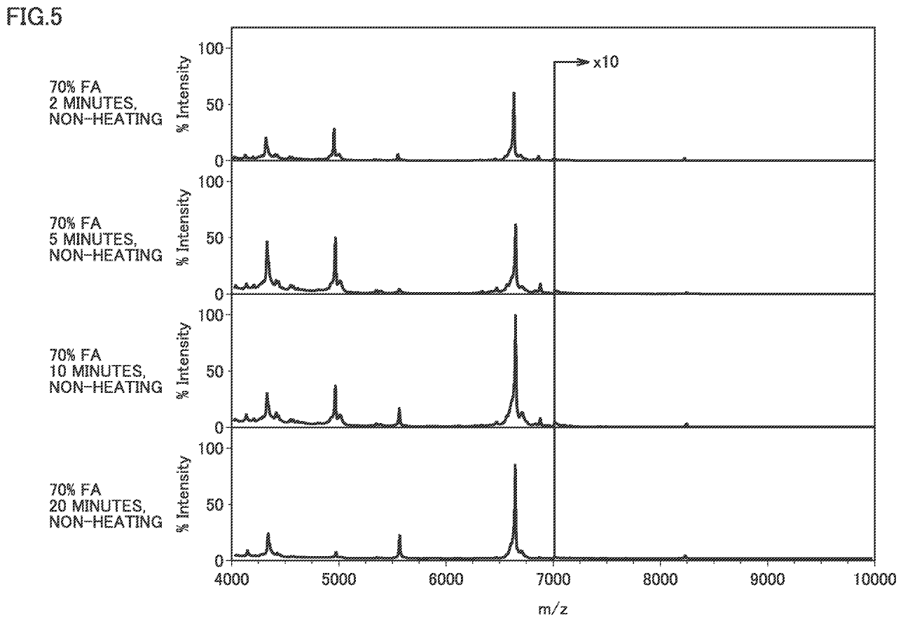

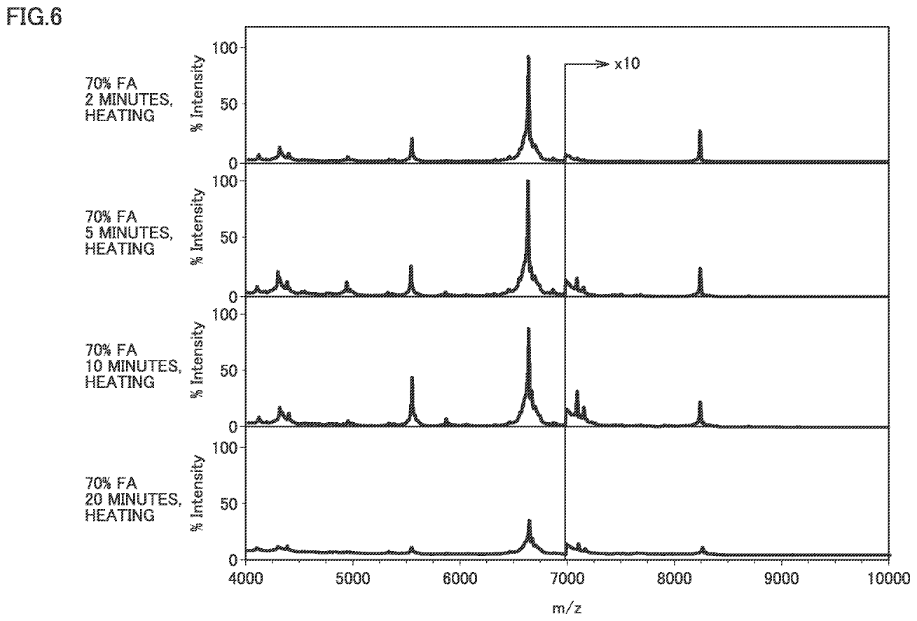

(4) and (5) of the sample pretreatment of Experiment 2 were performed as follows.
(4) A supernatant was removed, and 50 μL of 70% formic acid was added thereto, and the resultant was mixed with a vortex mixer to prepare a second acidic solution. Thereafter, the second acidic solution was dispensed into eight microtubes.
(5) Four of the microtubes into which the solution had been dispensed were subjected to a formic acid treatment without heating (at room temperature) respectively for 2 minutes, 5 minutes, 10 minutes, and 20 minutes (FIG. 5). The remaining four microtubes were subjected to a formic acid treatment with heating at 50° C. respectively for 2 minutes, 5 minutes, 10 minutes, and 20 minutes (FIG. 6). Thereafter, the sample held in each tube was subjected to the following treatments.

Hereinafter, the formic acid treatment without heating will be referred to also as the “non-heating formic acid treatment”, and the formic acid treatment with heating will be referred to also as the “heating formic acid treatment”.

FIG. 5 is a diagram illustrating mass spectra obtained by performing the non-heating formic acid treatment. FIG. 6 is a diagram illustrating mass spectra obtained by performing the heating formic acid treatment. In FIG. 5 and FIG. 6, the abscissa indicates the m/z. The ordinate of FIG. 5 indicates the relative intensity obtained by assuming that one peak having the highest intensity in the four mass spectra of FIG. 5 is 100%. The ordinate of FIG. 6 indicates the relative intensity obtained by assuming that one peak having the highest intensity in the four mass spectra of FIG. 6 is 100%. In FIG. 5 and FIG. 6, in a region of a m/z of 7000 or more, data is shown with 10-fold signal intensities.

Referring to FIG. 5, in the region of the m/z of 7000 or more, few peaks were detected even when the 10-fold intensities were shown. On the other hand, referring to FIG. 6, peaks were detected also in the region of the m/z of 7000 or more.

Hereinafter, in the description referring to FIG. 6, a mass spectrum obtained as a result of performing the heating formic acid treatment for X minutes will be referred to also as the “X minutes heating mass spectrum”.

Referring to FIG. 6, peak intensities in the vicinity of m/zs of 7000 to 7200 were increased by several or more times in the 5 minutes heating mass spectrum and the 10 minutes heating mass spectrum as compared with that in the 2 minutes heating mass spectrum. For example, as for a peak in the vicinity of a m/z of 7100, substantially no peak was detected in the 2 minutes heating mass spectrum, but definite peaks were detected in each of the 5 minutes heating mass spectrum and the 10 minutes heating mass spectrum.

On the other hand, in the 20 minutes heating mass spectrum, peak intensities were lower than in the 5 minutes heating mass spectrum and the 10 minutes heating mass spectrum. It is presumed, from these results, that there is a possibility that a protein had been degraded because formic acid was excessively reacted.

In other words, according to the results of Experiment 2, the intensities of the peaks corresponding to the m/zs of 7000 or more in each mass spectrum was improved by performing the heating formic acid treatment for 2 minutes or more and less than 20 minutes with formic acid added to the sample as compared with when the non-heating formic acid treatment was performed. Besides, also in the mass spectrum obtained by performing the heating formic acid treatment, the number and the intensities of peaks were further improved in the vicinity of the m/zs of 7000 to 7200 when the heating formic acid treatment was performed particularly for 5 minutes or more and 10 minutes or less.

### (4-3. Experiment 3)

Experiment 3 is an experiment showing change, caused by heating performed in a formic acid treatment, of the intensity of a peak corresponding to a cytoplasmic component.

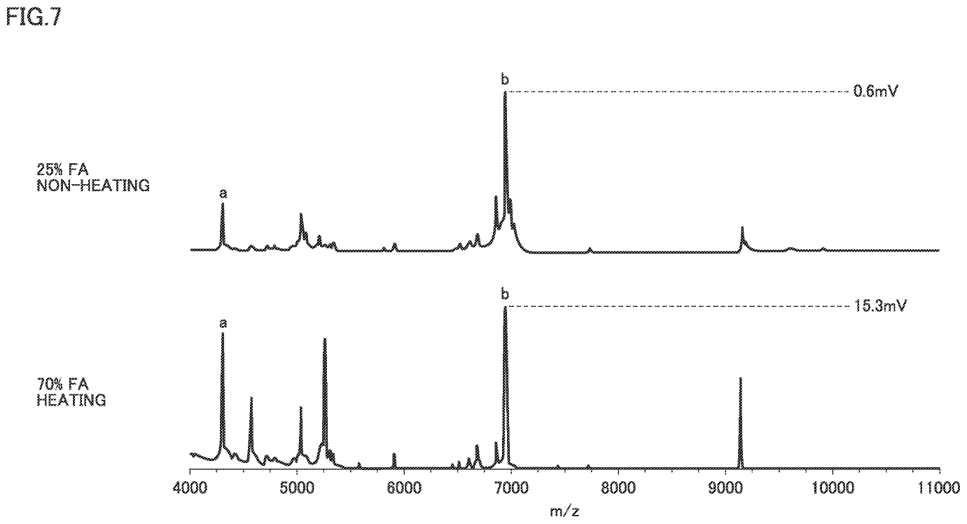

A sample pretreatment of Experiment 3 was performed as follows.
(1) 1 g of natto (fermented soybeans) was dispersed, in a microtube, in 9 mL of sterile physiological saline to be mixed with a vortex mixer, and the resultant was centrifuged to obtain a supernatant.
(2) The supernatant was smeared on a standard agar medium to be cultured at 30° C. for 24 hours, and thus, a bacterium isolated from natto was obtained.
(3) A colony of the bacterium isolated from natto was dispersed in ultrapure water in a microtube to obtain an OD of about 1, and the resultant was dispensed by 100 μL into two microtubes. These were centrifuged to remove a supernatant, and 100 μL of 25% formic acid and 100 μL of 70% formic acid were respectively added thereto. The sample to which 25% formic acid had been added was allowed to stand still at room temperature for 5 minutes, and then dropped in an amount of 1 μL onto a sample plate to be dried, and 1 μL of a matrix solution (CHCA solution) was dropped thereon to be dried (upper portion of FIG. 7). The sample to which 70% formic acid had been added was heated at 50° C. for 5 minutes, and then dropped in an amount of 1 μL onto a sample plate to be dried, 1 μL of a matrix solution (CHCA solution) was dropped thereon to be dried, and the resultant was subjected to measurement by MALDI-MS (lower portion of FIG. 7).
(4) After drying formic acid, a matrix solution (CHCA solution) was dropped onto the resultant to be dried, and the matrix dried product thus obtained was subjected to measurement by MALDI-MS.

FIG. 7 is a diagram illustrating change, caused by heating, of the intensity of a ribosomal protein peak obtained as a result of Experiment 3. In FIG. 7, the abscissa indicates the m/z. The ordinate indicates the relative intensity obtained by assuming that one peak having the highest intensity in the mass spectra of FIG. 7 is 100%. In the upper portion of FIG. 7, a peak having the highest intensity is a peak b, and the intensity is 0.6 mV. In the lower portion of FIG. 7, a peak having the highest intensity is a peak b, and the intensity is 15.3 mV, which is 25 times or more as high as 0.6 mV. In other words, even based on the peak having the highest intensity obtained by the non-heating formic acid treatment, the intensity 25 times or more as high was obtained in the heating formic acid treatment.

Besides, it is understood that a ratio of a peak a based on the peak b was about twice of that obtained in the heating formic acid treatment.

The peak a is a peak of one of ribosomal proteins most frequently used in determination of a microorganism. More specifically, it corresponds to L36 protein, that is, one of ribosomal proteins. On the other hand, the peak b was not attributed to any proteins contained in a cytoplasm. It is presumed, based on these results, that the lysis of the microorganism was accelerated through the heating formic acid treatment, and hence cytoplasmic components including ribosomal proteins could be efficiently extracted from the bacterial cell.

According to the results of Experiment 3, the intensity of a peak of a ribosomal protein, that is, a useful biomarker in determination of a microorganism, was improved by the heating formic acid treatment. In other words, it is presumed that the efficiency of determining a microorganism is improved by the heating formic acid treatment.

### (4-4. Experiment 4)

Experiment 4 shows change of the peak intensity caused by heating performed in a formic acid treatment in Bacillus subtilis subsp. subtilis NBRC 13719T.

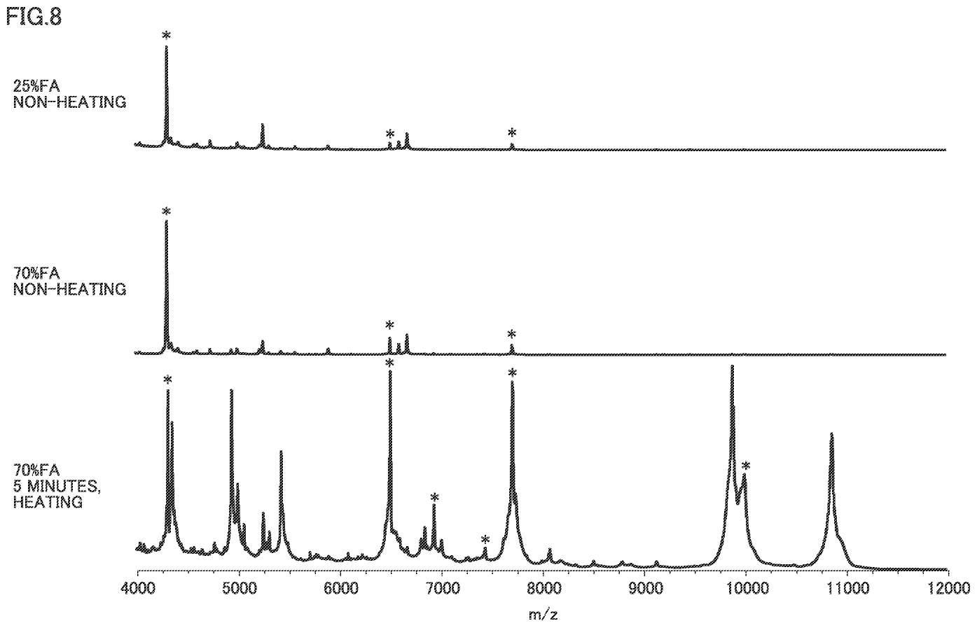

FIG. 8 is a diagram illustrating the results of Experiment 4, and illustrates a mass spectrum obtained by a 25% formic acid treatment performed without heating, a mass spectrum obtained by a 70% formic acid treatment performed without heating, and a mass spectrum obtained by a 70% formic acid treatment performed with heating for 5 minutes. In FIG. 8, the abscissa indicates the m/z. The ordinate indicates the relative intensity obtained by assuming that one peak having the highest intensity in all the mass spectra of FIG. 8 is 100%.

Referring to FIG. 8, it is understood that there is no large difference between the mass spectrum obtained by the 25% formic acid treatment performed without heating and the mass spectrum obtained by the 70% formic acid treatment performed without heating, but that only in the mass spectrum obtained by the 70% formic acid treatment performed with heating, the number and intensities of peaks were largely increased.

In particular, in peaks corresponding to ribosomal proteins (asterisked peaks), the improvement of the number and intensities of the peaks was remarkable. For example, when the 70% formic acid treatment was performed with heating, a peak in the vicinity of a m/z of 6500 exhibited an intensity about 12 times or more as high as that exhibited when the formic acid treatment was performed without heating. Similarly, a peak in the vicinity of a m/z of 7700 exhibited an intensity about 18 times or more.

According to the results of Experiment 4, peaks of a larger number of proteins could be detected with higher intensities by performing the heating formic acid treatment. In particular, the improvement of the intensity was remarkable in peaks of ribosomal proteins. In determination of a microorganism, a biomarker that is a prescribed type of protein is used, and therefore, the determination efficiency for a microorganism can be expected to be improved by the heating formic acid treatment.

### (4-5. Experiment 5)

Experiment 5 is an experiment showing the effect, in a large number of types of microorganisms, of heating performed in a formic acid treatment. Specifically, the experiment was performed on a group of microorganisms belonging to the phylum Actinobacteria including actinomycetes, which generally have a gram-positive cell wall, and are known to be difficult to be lysed.

A sample pretreatment of Experiment 5 was performed as follows.
(1) First, as the group of microorganisms belonging to the phylum Actinobacteria, Agromyces rhizosphaerae 14 (NBRC 16236), Arthrobacter globiformis 168 (NBRC 12137), Bifidobacterium longum E194bk (JCM 1217), Brachybacterium conglomeratum 5-2 (NBRC 15472), Corynebacterium glutamicum 534 (NBRC 12168), Glycomyces algeriensis LLR-39Z-86 (NBRC 103888), Glycomyces arizonensis DPL-G-76 (NBRC 103886), Glycomyces harbinensis LL-DO5139 (NBRC 14487), Janibacter limosus HKI 83 (NBRC 16128), Microlunatus phosphovorus NM-1 (NBRC 101784), Nocardioides simplex AJ 1420 (NBRC 12069), Paenarthrobacter aurescens 579 (NBRC 12136), Paenarthrobacter histidinolovorans (NBRC 15510), Phycicoccus duodecadis (NBRC 12959), and Streptomyces griseus C1 (NBRC 12875) were purchased from National Institute of Technology and Evaluation Biological Resource Center, or Riken Biological Resource Center, and were liquid-cultured with specified media.
(2) The thus cultured microorganism was dispensed, and a sample having been subjected to the in-tube non-heating method as a control experiment was subjected to mass spectrometry.
(3) The cultured microorganism was dispensed, and an experiment was performed by employing the pretreatment method of the present embodiment. Specifically, a sample having been subjected to the in-tube heating method including the in-tube heating treatment (50° C., 5 minutes) was subjected to mass spectrometry.

Next, the results of Experiment 5 will be described.

First, the results of the control experiment employing the conventional pretreatment method will be described. In Agromyces rhizospherae NBRC16236, Arthrobacter globiformis NBRC12137, Glycomyces algeriensis NBRC103888, Glycomyces arizonensis NBRC103886, Glycomyces harbinensis NBRC14487, Janibacter limosus NBRC16128, Nocardioides simplex NBRC12069, Paenarthrobacter histidinolovorans NBRC15510, Streptomyces griseus NBRC12875 and the like, there were, in some samples, only five or less peaks that corresponded to, within 200 ppm, ribosomal proteins estimated based on theoretical values of genome information in some cases, which suggested that there was a possibility that ribosomal proteins were not sufficiently extracted by the in-tube formic acid treatment. On the other hand, in the other strains, peaks corresponding to about 6 to 20 ribosomal proteins within 200 ppm were obtained, and ribosomal proteins were favorably extracted by the in-tube formic acid treatment.

On the other hand, when the pretreatment method of the present embodiment was employed, in all the tested strains described above, peaks corresponding to about 6to 20 ribosomal proteins within 200 ppm were obtained.

These results reveal that the formic acid treatment accompanied by a heating treatment is effective for not only a group of microorganisms, such as E. coli, that have the gram-negative cell wall structure, and from which an intracellular composition is comparatively easily extracted but also a group of microorganisms, such as those belonging to the phylum Actinobacteria, having a strong cell wall. In this manner, it was suggested that the pretreatment method of the present embodiment is a highly versatile method by which lysis of microorganisms belonging to various taxonomic groups can be encouraged under the same conditions.

### (4-6. Experiment 6)

Experiment 6 is an experiment in which Escherichia coli of a gram-negative bacterium, and Janibacter limosus of a gram-positive bacterium were subjected to the formic acid treatment under a plurality of temperature conditions.

A sample pretreatment of Experiment 6 was performed as follows.
(1) As bacterial cells to be used in Experiment 6, Escherichia coli (ATCC 700926) having been shake-cultured at 37° C. overnight in a GAM liquid medium prepared with GAM broth (Shimadzu Diagnostics 05422), and Janibacter limosus (NBRC 16128) having been cultured at 30° C. for 2 days in 802 agar medium were used. As for Janibacter limosus, 50 μL of a culture fluid containing the bacterial cell was collected, and added to a 96 well U bottom cell culture plate (FALCON 353077). Then, the culture fluid thus added was measured with SpectraMax iD3, and a corresponding liquid medium was added to each well to obtain an OD (optical density) value of about 0.4 to 0.6, and thus, a bacterial liquid was obtained. As for Escherichia coli, the bacterial cell collected from the 802 agar medium was suspended in distilled water to be prepared to obtain an OD value equivalent to that of Janibacter limosus (about 0.4 to 0.6).
(2) A 1.5 mL tube was charged with 400 μL of the bacterial liquid thus obtained, and centrifuged with a centrifugal machine (15,000 rpm, room temperature, 5 minutes), and a supernatant was discharged from the tube. To the tube from which the supernatant had been removed, 250 μL of pure water was added to resuspend a pellet of the bacterial cell. Thereafter, 750 μL of ethanol (99.5%) was added to the tube to obtain a final concentration of the ethanol of 75% (v/v). The bacterial cell was washed within the tube, and then centrifuged with a centrifugal machine (15,000 rpm, room temperature, 5 minutes), and a supernatant was removed from the tube. Thereafter, the resultant was air-dried to remove the ethanol from the tube. Then, 60 μL of ultrapure water (FUJIFILM 214-01301) was added to the tube to suspend the air-dried pellet of the bacterial cell. 140 μL of formic acid (FUJIFILM 067-04531) was added, to be mixed, to the tube to a final concentration of formic acid of 70% (v/v) (contacting process). The thus mixed suspension was dispensed into a 0.2 mL tube by 10 μL. Respective dispensed suspensions were heated, by using VeriFlex function of MiniAmp Plus Thermal Cycler (Applied Biosystems), respectively at 20° C., 40° C., 50° C., and 60° C. for 5 minutes (heating process). It is noted that 20° C. is usually a temperature equal to or lower than room temperature or approximate to room temperature, and hence does not correspond to “heating”, but is described as “heating” for convenience sake in Experiment 6. After the heating, the tubes holding the suspensions were kept cold on ice. To each of the heated samples (suspensions), 10 μL of acetonitrile (FUJIFILM 012-19851) was added to be mixed. 1 μL of each sample was placed on a spot of MALDI sample plate FlexiMass-DS (SHIMADZU BIOTECH TO-430) to be air-dried. On the other hand, to a solution of 50% acetonitrile (v/v) and 1% trifluoroacetic acid (Wako 206-1731) (v/v) in ultrapure water, α-cyano-4-hydroxycinnamic acid (CHCA) (TCI C1768) was added to a final concentration of 10 mg/mL, whereby obtaining a matrix solution (prepared as needed). Onto a spot in which the air-dried sample was set, 1 μL of the matrix solution was added, the matrix solution and the sample were mixed by pipetting, and the resultant was further air-dried. Through these processings, each MALDI sample plate having the sample provided thereon was prepared.
(3) Each of the MALDI sample plates thus prepared was inserted into MALDI-8020 manufactured by Shimadzu Corporation for performing the measurement under the following conditions: Acquire tab was set to mass range: 2000 to 20000, accumulate 5 shot(s) was set to @200 Hz blast shot, and Profiles was set to 100 profiles (20220601_Microbio). Process tab was set to Subtract baseline using filter width 500, Smoothing method was set to Gaussian, Peak width was set to 80, Peak delimiter method was set to Threshold Apex, and Threshold offset and response was set to 0.015 mV and 1.2000 mV (20221223 microbio). For calibration of MALDI-8020, Escherichia coli was used as a standard sample. Specifically, the m/z axis was calibrated by using a plurality of peaks known to be observed in Escherichia coli. More specifically, a suspension of Escherichia coli prepared with 50% acetonitrile (v/v) was used in the calibration by using peaks corresponding to 4365.3 Da, 6316.2 Da, 6411.6 Da, 7158.8 Da, 7274.5 Da, 8369.8 Da, 8994.3 Da, 9060.4 Da, 10138.6 Da, 10300.1 Da, 10694.4 Da, 11450.3 Da, 12227.3 Da, and 12770.6 Da (Ec221122).
(4) The peaks obtained from the samples measured with MALDI-8020 were analyzed to calculate the total number of peaks and the number of peaks of ribosomal proteins. At this point, GPMsDB-tk v1.0.1 (https://github.com/ysekig/GPMsDB-tk) was used for the MALDI data analysis.

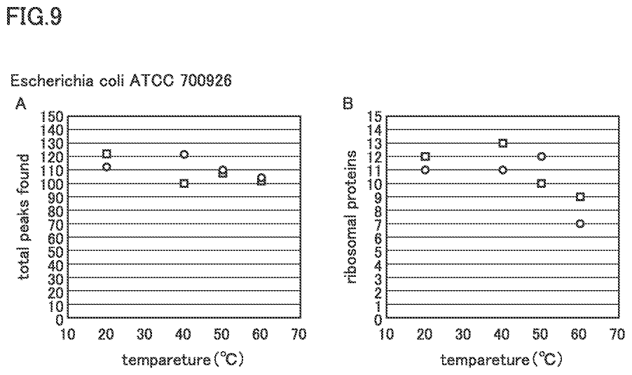

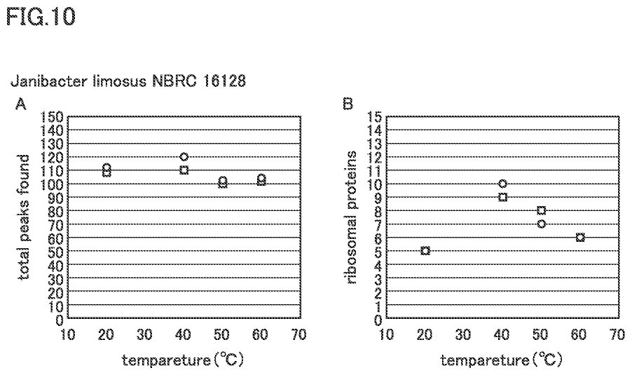

The results of Experiment 6 are illustrated in FIG. 9 and FIG. 10. FIG. 9 is a diagram illustrating the numbers of peaks obtained when the formic acid treatment was performed on Escherichia coli under a plurality of temperature conditions. FIG. 10 is a diagram illustrating the numbers of peaks obtained when the formic acid treatment was performed on Janibacter limosus under a plurality of temperature conditions. The abscissas of FIG. 9 and FIG. 10 indicate the temperature (° C.). The ordinates of FIG. 9A and FIG. 10A indicate the number of total peaks found, that is, the total number of peaks detected in the mass spectrum. The ordinates of FIG. 9B and FIG. 10B indicate the number of peaks, among all the peaks detected in the mass spectrum, identified as ribosomal proteins based on the m/zs thereof (ribosomal proteins). The results obtained by performing the experiment on each of Escherichia coli and Janibacter limosus twice under each of the respective temperature conditions are shown with square and circular markers in FIG. 9 and FIG. 10.

Referring to FIG. 9, in Escherichia coli whose cells can be easily disrupted, when the formic acid treatment was performed at 20° C., 40° C., and 50° C., substantially the same number (10 to 13) of ribosomes were detected. On the other hand, when the formic acid treatment was performed under a high temperature condition (of, for example, 60° C.), the number of detected ribosomal proteins was remarkably reduced. The results of FIG. 9 indicate that proteins derived from a bacterial cell can be detected in gram-negative bacteria such as Escherichia coli by the formic acid treatment performed under various temperature conditions. It is revealed, in particular, that the formic acid treatment can be performed with minimizing protein degradation under a temperature condition of 20° C. or more to about 50° C.

Referring to FIG. 10, in Janibacter limosus whose cells are comparatively difficult to be disrupted, the number of detected ribosomal proteins were larger when the formic acid treatment was performed at 40° C. to 50° C. as compared with when the formic acid treatment was performed at 20° C. The results of FIG. 10 indicate that the formic acid treatment performed under a high temperature condition (of about 40° C. to 50° C.) for a short period of time (of 5 minutes) is effective for disrupting cells of gram-positive bacteria such as Janibacter limosus.

As described above, when the formic acid treatment was performed at about 40° C. to 50° C., the number of detected ribosomal proteins was not largely reduced even in Escherichia coli whose cells can be easily disrupted. Accordingly, the formic acid treatment performed at about 40° C. to 50° C. is a treatment condition not causing large damage in MALDI measurement of a microorganism whose cells are easily disrupted. As described so far, the heating in the formic acid treatment performed at about 40° C. to 50° C. does not reduce the number of peaks of cytoplasmic components (proteins or the like present in the cytoplasm) in cells of a microorganism whose cells are easily disrupted, such as Escherichia coli, and encourages cell disruption in bacterial strains whose cells are difficult to be disrupted, such as Janibacter limosus. Accordingly, it is revealed that the formic acid treatment performed for 5 minutes with heating at about 40° C. to 50° C. is a pretreatment method commonly used for a bacterium whose cells are easily disrupted, and for a bacterium whose cells are difficult to be disrupted. In other words, the heating formic acid treatment performed for 5 minutes at about 40° C. to 50° C. is a pretreatment method applicable to a wide range of bacterial strains by which peaks of proteins present in the cytoplasm can be efficiently detected.

It is noted that the detection efficiency of peaks of proteins present in the cytoplasm using formic acid relates to the temperature and the time. For example, when the formic acid treatment is performed at 20° C. for a long period of time, or when the formic acid treatment is performed at 60° C. for a short period of time (of, for example, 1 minute), the effect equivalent to that obtained by performing the formic acid treatment at 50° C. for 5 minutes can be probably obtained. It was revealed that by adjusting the heating time and temperature in this manner, cells can be disrupted with excessive degradation of protein suppressed.

### (4-7. Experiment 7)

Experiment 7 is an experiment showing the effect of the heating performed in the formic acid treatment on a filamentous fungus that is difficult to be lysed (Aspergillus kawachii NBRC 4308).

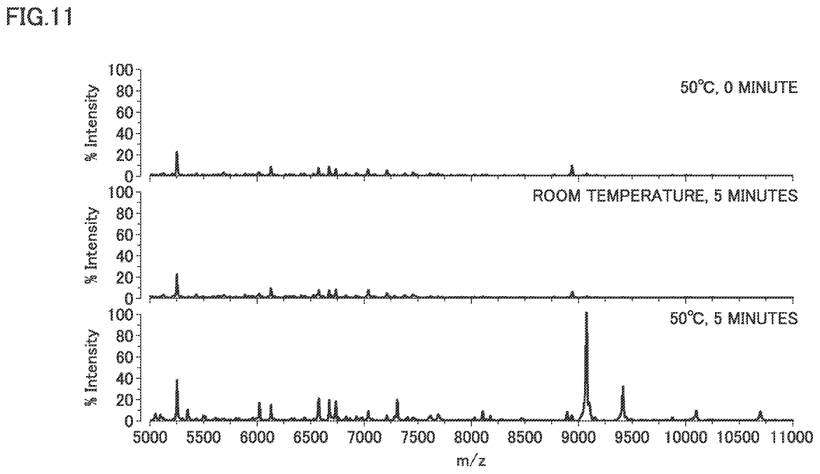

A sample pretreatment of Experiment 7 was performed as follows.
(1) A filamentous fungus (Aspergillus kawachii NBRC 4308) was cultured in a potato dextrose agar medium.
(2) The resultant hypha was scraped with a cotton swab, and dispersed in 1000 μL of water to obtain a dispersion. 200 μL of the thus obtained dispersion was dispensed into three tubes. To each of the three tubes, 800 μL of ethanol was further added. Each tube was centrifuged to remove a supernatant, and 50 μL of 70% formic acid was added to a resultant precipitate (a pellet of the bacterial cell) to be redispersed (contacting process).
(3) After (2), one of the tubes (a sample corresponding to data of (50° C., 0 minutes) of FIG. 11) was heated to 50° C., and immediately after, 50 μL of acetonitrile was added thereto to be mixed, and the resultant was centrifuged to obtain a supernatant. 0.5 μL of the supernatant was dropped onto a sample plate to be dried. After the drying, 1 μL of a CHCA solution was dropped thereon to be dried again. The CHCA solution was prepared by dissolving CHCA to 10 mg/mL in an aqueous solution containing 35% acetonitrile containing 1% trifluoroacetic acid, and 15% ethanol.
(4) After (2), another of the tubes (a sample corresponding to data of (room temperature, 5 minutes) of FIG. 11) was allowed to stand still at room temperature (23° C.) for 5 minutes. The other tube (a sample corresponding to data of (50° C., 5 minutes) of FIG. 11) was heated at 50° C. for 5 minutes (heating process). Thereafter, to each of these two tubes, 50 μL acetonitrile was added to be mixed, and the resultant was centrifuged to obtain a supernatant. 0.5 μL of the supernatant was dropped onto a sample plate to be dried. After the drying, 1 μL of the CHCA solution was dropped thereon to be dried again.
(5) The samples having been dried after adding the CHCA solution in (3) or (4) were measured with MALDI-8020.

FIG. 11 is a diagram illustrating mass spectrum change caused by performing the heating formic acid treatment on Aspergillus kawachii. Referring to FIG. 11, a large number of peaks could be detected in the filamentous fungus by performing the heating formic acid treatment at 50° C. for 5 minutes. According to the results of Experiment 7, it was revealed that the heating formic acid treatment is a highly versatile method effective also for Aspergillus kawachii.

### 5. Conclusion

As described so far, according to the pretreatment method of the present embodiment, even in a microorganism having a strong cell wall, a mass spectrum having a high peak intensity can be obtained by heating a sample in contact with an acidic solution. In particular, the number or intensities of peaks of ribosomal proteins, that is, biomarkers useful for determination of a microorganism, can be improved. When it was changed, with keeping all the other conditions excluding the heating, whether or not the heating was performed, the number and intensities of peaks were improved. Therefore, the improvement of the number and intensities of peaks is caused probably because lysis with the acidic solution was appropriately promoted by the heating. Besides, as a result of the improvement of the number and intensities of peaks, microorganism determination efficiency is improved.

In particular, the pretreatment method of the present embodiment was effective for the improvement of the number and intensities of peaks also in a group of mycolic acid-producing bacteria, that is, a group of bacteria significant in medical and clinical research.

Usually, when a cell is excessively heated, it is presumed that protein may be degraded into a large number of fragments. In this case, peaks corresponding to the large number of fragments are detected in a mass spectrum, and it is apprehended that it may be difficult to detect a peak corresponding to the protein before degradation. In the pretreatment method of the present embodiment, cell wall destruction is accelerated by a combination of an acidic solution in a prescribed concentration and appropriate heating, and thus, it is possible that a protein contained in the cytoplasm or the like is not excessively degraded. Therefore, a peak of a ribosomal protein serving as a biomarker can be detected with high accuracy.

It is noted that there is a possibility that the three-dimensional structure of a protein molecule may be slightly changed (modified) also in the pretreatment method of the present embodiment, but the mass itself of the protein molecule does not change even when it is modified, and therefore, there arises no problem in mass spectrometry.

The pretreatment method of the present embodiment has a characteristic that it is easily performed by a user. When the on-plate heating method is employed, after dispersing the bacterial cell in the first acidic solution, the heating can be performed on each sample plate. Alternatively, when the in-tube heating method is employed, the heating can be performed with a PCR device that is installed in most of laboratories handling microorganisms. Besides, since a bead is not used, the handling is easy, and the method is secure because beads are never scattered in a device such as a mass spectrometer. Since the method is applicable to a wide range of microorganisms including a microorganism having a strong cell wall, there is no need to study a pretreatment method depending on the type of microorganism. More particularly, in a wide range of types of microorganisms, the effect of improving peak intensities can be obtained under the comparatively common conditions (of, for example, heating for 2 to 20 minutes). In consideration of these characteristics, the pretreatment method of the present embodiment is useful also in that a wide range of users can easily perform it.

In particular, the pretreatment method of the present embodiment is completed by only a simple liquid operation and heating, and therefore, some or all of steps can be easily automated with a machine. More particularly, the method can be easily incorporated into a pretreatment device. Besides, for the same reason, when a plurality of containers respectively holding a prescribed type of microorganism are prepared to simultaneously test a plurality of conditions selected from the above- described comparatively common conditions, it is easy to obtain an optimum condition for obtaining the highest peak intensity through one experiment, and to obtain a mass spectrum under the optimum condition.

Besides, although it was mainly described in the above examples that the intensity of a peak corresponding to an intracellular component of a microorganism can be improved by the pretreatment method of the present embodiment, it is obvious that the pretreatment method of the present embodiment is applicable to a biological sample containing a cell of an organism except for a microorganism. For example, the method is useful also for extracting an intracellular component by destroying a cell wall and/or a cell membrane of a cell except for that of a microorganism.

Besides, although it was described in the above examples that a cytoplasmic component is extracted by contacting an organic acid and a first acidic solution, and heating the resultant in the pretreatment method of the present embodiment, a first acidic solution containing an inorganic acid may be used as long as effects similar to those of the pretreatment method of the present embodiment are exhibited. The examples of the first acidic solution containing an organic acid are not limited to those described above as long as effects similar to those of the pretreatment method of the present embodiment are exhibited. It is noted that a first acidic solution that exhibits the effects of the present application refers to, for example, an acidic solution that improves the intensity of a peak of a cytoplasmic component when subjected to a heating acid treatment. It is noted that a first acidic solution that does not exhibit the effects of the present application refers to, for example, an acid that cannot sufficiently destroy a cell wall, an acid that degrades a protein to an extent that detection of a peak of a cytoplasmic component is obstructed, and/or an acidic solution that causes a problem in a step except for the cytoplasm extraction. For example, an acidic solution containing hydrochloric acid generally has a possibility of melting a sample plate containing stainless steel, and hence is probably unsuitable to be used in the on-plate heating treatment of the present embodiment. On the other hand, an acidic solution containing hydrochloric acid is usable in the in-tube heating treatment performed within an acid-resistant container (for example, a container made of glass or an acid-resistant resin). In this case, the sample is preferably washed after the in-tube heating treatment. In this manner, an acid used in the pretreatment method of the present embodiment can be optionally selected in a range conceived by those skilled in the art.

### Aspects

Those skilled in the art will understand that the above-described plurality of exemplifying embodiments are specific examples of the following aspects.
(Item 1) A pretreatment method according to one aspect is a method of pretreatment of a sample containing a cell for mass spectrometry, including contacting the cell with a first acidic solution containing an organic acid; and extracting a cytoplasmic component of the cell by heating the cell in contact with the first acidic solution.

According to the pretreatment method of item 1, extraction efficiency of the cytoplasmic component is improved. Therefore, the intensity of a peak corresponding to the cytoplasmic component in a mass spectrum is improved. In other words, through the pretreatment of a sample, for mass spectrometry, containing a cell, the intensity of a peak of a cytoplasmic component can be improved.
(Item 2) In the pretreatment method according to item 1, the contacting includes preparing a second acidic solution that is a mixture solution of the sample and the first acidic solution. The extracting includes heating the second acidic solution.

According to the pretreatment method of item 2, the cell can be dispersed in the first acidic solution by mixing the sample and the first acidic solution. Thus, the cell and the first acidic solution can definitely contact with each other, and hence the efficiency of the treatment with the first acidic solution on the cell is improved.
(Item 3) In the pretreatment method according to item 1 or 2, the organic acid contains at least one of formic acid, trifluoroacetic acid, and acetic acid.

According to the pretreatment method of item 3, the pretreatment method of the present embodiment can be performed by utilizing these acids easily available for and familiar in handling to users of the research and clinical fields performing mass spectrometry of microorganisms. Besides, these acids are useful also in that those skilled in the art experientially know that these acids do not harmfully affect a measurement result even when used in a pretreatment of mass spectrometry.
(Item 4) In the pretreatment method according to any one of items 1 to 3, a concentration of the organic acid is 50 vol % or more and 90 vol % or less based on the first acidic solution or the second acidic solution.

According to the pretreatment method of item 4, the pretreatment method can be performed using the acidic solution at the concentration.
(Item 5) In the pretreatment method according to item 4, the concentration of the organic acid is 65 vol % or more and 75 vol % or less based on the first acidic solution or the second acidic solution.

According to the pretreatment method of item 5, the pretreatment method of the present embodiment can be performed using the acidic solution at the concentration.
(Item 6) In the pretreatment method according to any one of items 1 to 5, the first acidic solution contains water or an organic solvent.

According to the pretreatment method of item 6, the pretreatment method of the present embodiment can be performed by using the first acidic solution prepared with any of these solvents used.
(Item 7) In the pretreatment method according to any one of items 1 to 6, a heating time is 2 minutes or more and less than 20 minutes.

According to the pretreatment method of item 7, the pretreatment method of the present embodiment can be performed by performing the heating for the time period.
(Item 8) In the pretreatment method according to item 7, the heating time is 5 minutes or more and 10 minutes or less.

According to the pretreatment method of item 8, the pretreatment method of the present embodiment can be performed by performing the heating for the time period.
(Item 9) In the pretreatment method according to any one of items 1 to 8, a heating temperature is 30° C. or more and 75° C. or less.

According to the pretreatment method of item 9, the pretreatment method of the present embodiment can be performed by performing the heating at the temperature.
(Item 10-1) In the pretreatment method according to item 9, the heating temperature is 40° C. or more and 60° C. or less.
(Item 10-2) In the pretreatment method according to item 9, the heating temperature is 35° C. or more and 55° C. or less.

According to the pretreatment method of item 10-1 or 10-2, the pretreatment method of the present embodiment can be performed by performing the heating at the temperature.
(Item 11) In the pretreatment method according to item 2, the heating the second acidic solution includes heating the second acidic solution placed on a sample plate for mass spectrometry, or heating the second acidic solution that is held in a container before the second acidic solution being placed on the sample plate.

In the pretreatment method according to item 11, the second acidic solution can be heated by any of the two simple methods. A user can select a preferred one of the two simple methods.
(Item 12) In the pretreatment method according to any one of items 1 to 11, the cell is a cell of a microorganism.

According to the pretreatment method of item 12, the pretreatment method of the present embodiment can be performed also in mass spectrometry of a microorganism in the fields of clinical microorganism analysis, food safety test, and the like.
(Item 13) In the pretreatment method according to any one of items 1 to 12, the cell is a cell having a cell wall.

According to the pretreatment method of item 13, the cell wall can be destroyed by the pretreatment method of the present embodiment to improve the intensity of a peak of a cytoplasmic component.
(Item 14) In the pretreatment method according to any one of items 1 to 13, the mass spectrometry is mass spectrometry by a matrix-assisted laser desorption/ionization method.

According to the pretreatment method of item 14, the mass spectrometry can be performed by employing the MALDI method suitable for determination of a microorganism by mass spectrometry of the microorganism.
(Item 15) A mass spectrometry method including obtaining a mass spectrum by performing mass spectrometry, by a matrix-assisted laser desorption/ionization method, on the sample having been pretreated by the pretreatment method according to any one of items 1 to 14.

It should be regarded that the embodiments disclosed herein are not limiting but illustrative in all points. The scope of the present invention is not limited by the description given above but limited by the scope of appended claims, and is intended to encompass equivalence of the scope of appended claims, and all modifications made within the scope.

### REFERENCE SIGNS LIST

1 Analysis device; 10 Control unit; 11 Processing part; 12 Storage part; 13 Input/output part; 20 Measurement unit; 21 Ionization part; 22 Ion acceleration part; 23 Mass separation part; 24 Detection part; 111 Device control part; 113 Mass spectrum analysis part; 114 Determination part; 131 Input part; 132 Output part; 133 Communication part; 221 Accelerating electrode; 231 Flight tube.

1. A method of pretreatment of a sample containing a cell for mass spectrometry, comprising:
preparing a suspension by mixing the sample and a first acidic solution containing an organic acid; and
extracting a cytoplasmic component of the cell by heating the suspension that is held in a container before the suspension being placed on a sample plate for mass spectrometry.

2. (canceled)

3. The pretreatment method according to claim 1, wherein the organic acid contains at least one of formic acid, trifluoroacetic acid, and acetic acid.

4. The pretreatment method according to claim 2, wherein a concentration of the organic acid is 50 vol % or more and 90 vol % or less based on the first acidic solution or the suspension.

5. The pretreatment method according to claim 4, wherein the concentration of the organic acid is 65 vol % or more and 75 vol % or less based on the first acidic solution or the suspension.

6. The pretreatment method according to claim 1, wherein the first acidic solution contains water or an organic solvent.

7. The pretreatment method according to claim 1, wherein a heating time is 2 minutes or more and less than 20 minutes.

8. The pretreatment method according to claim 7, wherein the heating time is 5 minutes or more and 10 minutes or less.

9. The pretreatment method according to claim 1, wherein a heating temperature is 30° C. or more and 75° C. or less.

10. The pretreatment method according to claim 9, wherein the heating temperature is 35° C. or more and 55° C. or less.

11. (canceled)

12. The pretreatment method according to claim 1, wherein the cell is a cell of a microorganism.

13. The pretreatment method according to claim 1, wherein the cell is a cell having a cell wall.

14. The pretreatment method according to claim 1, wherein the mass spectrometry is mass spectrometry by a matrix-assisted laser desorption/ionization method.

15. A mass spectrometry method comprising obtaining a mass spectrum by performing mass spectrometry, by a matrix-assisted laser desorption/ionization method, on the sample having been pretreated by the pretreatment method according to claim 1.
